(19) United States
(12) Patent Application Publication
(10) Pub. No.: US 2011/0021517 A1
(43) Pub. Date: Jan. 27, 2011

(54) ANTITUMOR COMBINATION COMPRISING A MORPHOLINYL ANTHRACYCLINE DERIVATIVE AND DEMETHYLATING AGENTS

(75) Inventors: Maria Cristina Geroni, Milan (IT); Olga Valota, Legnano (MI) (IT); Massimo Broggini, Milan (IT)

Correspondence Address: SCULLY SCOTT MURPHY & PRESSER, PC, 400 GARDEN CITY PLAZA, SUITE 300, GARDEN CITY, NY, 11530, US

(73) Assignee: NERVIANO MEDICAL SCIENCES S.R.L., Nerviano (IT)

(21) Appl. No.: 12/918,938

(22) PCT Filed: Feb. 25, 2009
(86) PCT No.: PCT/EP2009/052237

§ 371 Date: Oct. 7, 2010

(30) Foreign Application Priority Data
Feb. 26, 2008 (EP) 08102012.5

(51) Int. Cl. A61K 31/5377; A61P 35/00

(52) U.S. Cl. 514/231.5

(57) ABSTRACT

The present invention provides a combination of a morpholinyl anthracycline derivative, in particular a methoxy morpholino doxorubicin derivative, or a pharmaceutically acceptable salt thereof, and a demethylating agent, having an antineo-plastic effect. Also provided is the use of the said combinations in the treatment or prevention of metastasis, in the treatment of tumors, as well for reversing the resistance in cells resistant to such a methoxy morpholino doxorubicin derivative.

The present invention relates to the field of cancer treatment and provides an antitumor combination comprising a morpholinyl anthracycline derivative and a demethylating agent, endowed with a good antineoplastic effect.

Morpholinyl anthracyclines are known in the art as cytotoxic agents useful in antitumor therapy.

Cancers are a leading cause of death in humans; surgery, radiation and chemotherapy are the useful means to fight cancers. In particular, combined chemotherapy, designed to treat cancer by using more than one drug in combination or association, is a well-accepted modality of treatment of neoplastic diseases such as cancer. Several efforts have been and are still being undertaken in order to select antitumor combinations more and more active and safe to be administered to a patient suffering from a cancer. The increase of the antitumor efficacy of a known antitumor compound by administering the same in combination with one or more different antitumor drugs in order to reduce the toxic effects of the individual agents when used alone, and in some instances because the combination has greater efficacy than when either agent is used alone, is a strongly felt need in the field of anticancer therapy.

U.S. Pat. No. 4,672,057 discloses and claims a morpholinyl anthracycline derivative named nemorubicin, the pharmaceutically acceptable salts, preparation process, pharmaceutical compositions and medical uses thereof. A crystalline form of nemorubicin hydrochloride is described and claimed in WO2008006720 (Nerviano Medical Sciences Srl).

In particular, nemorubicin represents a therapeutic option in the treatment of a liver cancer, and nemorubicin administration ways are described and claimed in WO 00/15203 and WO 04/75904 (Nerviano Medical Sciences Srl).

WO 04/082579 and WO 00/066093 (Nerviano Medical Sciences Srl) are relating to a combined use of morpholinyl anthracycline derivatives with radiotherapy or another anticancer drug such as an alkylating agent, an antimetabolite, a topoisomerase I or topoisomerase II inhibitor or a Platinum derivative.

The present invention fulfils the need of improved cancer treatment by providing a combined administration of a morpholinyl anthracycline derivative or a pharmaceutically acceptable salt, with a demethylating agent, having a good antineoplastic effect.

The present invention provides new combinations of a morpholinyl anthracycline derivative with known pharmaceutical agents that are particularly suitable for the treatment of proliferative disorders, especially cancer. More specifically, the combinations of the present invention are very useful in therapy as antitumor agents and lack, in terms of both toxicity and side effects, the drawbacks associated with currently available antitumor drugs. Moreover, this combination is useful for treating also the tumours resistant to nemorubicin.

It is therefore an object of the present invention a combination comprising a morpholinyl anthracycline derivative having formula (I)

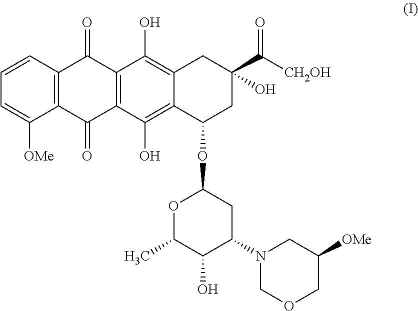

or a pharmaceutically acceptable salt thereof, and a demethylating agent, having an antineoplastic effect.

Another aspect provides a pharmaceutical composition comprising a combination according the invention admixed with a pharmaceutically acceptable carrier, diluent or excipient.

A further aspect relates to a combination according the invention for treating a proliferative disorder or for reversing the resistance. A still further aspect relates to a pharmaceutical product comprising a morpholinyl anthracycline as defined above and a demethylating agent, as a combined preparation for simultaneous, sequential or separate use for treating a proliferative disorder or for reversing the resistance.

Another aspect relates to the use of a morpholinyl anthracycline as defined above and a demethylating agent in the preparation of a medicament for treating a proliferative disorder or for reversing the resistance.

Another aspect relates to a method of treating a proliferative disorder, said method comprising simultaneously, sequentially or separately administering a morpholinyl anthracycline as defined above and a demethylating agent to a subject.

A still further aspect relates to the use of a morpholinyl anthracycline as defined above in the preparation of a medicament for the treatment of a proliferative disorder or for reversing the resistance, wherein said treatment comprises simultaneously, sequentially or separately administering a morpholinyl anthracycline as defined above and a demethylating agent.

In the present description, unless otherwise specified, the morpholinyl anthracycline derivative having formula (I) is nemorubicin, chemical names (8S-cis, 2″S)-7,8,9,10-tetrahydro-6,8,11-trihydroxy-8-(hydroxyacetyl)-1-methoxy-10-{[2,3,6-trideoxy-3-(2-methoxy-4-morpholinyl)-α-L-lyxo-hexopyranosyl]oxy}-5,12-naphthacenedione and 3′desamino-3′ [2(S)methoxy-4-morpholinyl] doxorubicin.

Nemorubicin, also known as 3′desamino-3′[2(S)methoxy-4-morpholinyl] doxorubicin, is a doxorubicin (DX) derivative different from classical anthracyclines, obtained with the substitution of the —NH₂ at position 3′ in the sugar moiety with a methoxymorpholinyl group.

As used herein, the term “nemorubicin” includes, unless otherwise specified, the morpholinyl anthracycline derivative of formula (I) and its pharmaceutically acceptable salts.

The term “pharmaceutically acceptable salt” refers to those salts retaining the biological effectiveness and properties of the parent compound. Such salts include acid addition salts obtained by reaction of the free base of the parent compound with inorganic acids such as hydrochloric, hydrobromic, nitric, phosphoric, sulphuric, and perchloric acid and the like; or with organic acids such as acetic, maleic, methanesulphonic, ethanesulfonic, tartaric, citric, succinic and the like.

Preferably, nemorubicin is in the form of its hydrochloride salt.

It has now been surprisingly found that the antitumor effect of a morpholinyl anthracycline derivative of formula (I) is greatly enhanced when it is administered in combination with a demethylating agent.

The effect of the combined administration is significantly increased both in cells sensitive and resistant to nemorubicin (synergic and additive effects) with respect to the effect obtained administering each drug as single agent.

The present invention provides, in a first aspect, a combination comprising a morpholinyl anthracycline derivative having formula (I) and demethylating agents. Demethylating agents, according to the present invention, are preferably selected from the group consisting of 5′aza-cytidine, 5-aza-2′-deoxycytidine (decitabine), zebularine [1-(β-D-ribofuranosyl(dihydro-pyrimidin-2-1)], L-methionine, inhibitors of histone deacetylase (HDAC) such as, for instance, valproic acid or trichostatin A, apicidine, hydralazine, procainamide (pronestyl), antisense oligonucleotides directed against DNA methyltransferase messeger RNA, their admixtures and derivatives thereof. Preferably, the demethylating agents to be used in the present invention are the following ones: decitabine, zebularine, valproic acid, trichostatin A, apicidine, and antisense oligonucleotides directed against DNA methyltransferase messeger RNA. The term “pharmaceutically effective amount” shall mean that amount of a drug or pharmaceutical agent that will elicit the biological or medical response of a tissue, system, animal or human that is being sought by a researcher or clinician. This amount can be a therapeutically effective amount.

The term “therapeutically-effective” is intended to qualify the amount of each agent for use in the combination therapy, which will achieve the goal of improvement in disease severity and the frequency of incidence over treatment of each agent by itself, and/or of amelioration of adverse side effects typically associated with alternative therapies.

The combined preparations according to the present invention are useful for the treatment of cancer. Preferably, the subject methods and compositions of the present invention may be used for the treatment of neoplasia disorders including benign, metastatic and malignant neoplasias, and also including acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of cancer. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has cancer, cardiovascular disease, or pain, inflammation and/or any one of the known inflammation-associated disorders.

The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

The subject pharmaceutical compositions may be administered to a patient in any acceptable manner that is medically acceptable including orally, parenterally or with locoregional therapeutic approaches such as e.g. implants.

Parenteral administration includes administering the constituents of the combined preparation by subcutaneous, intramuscular, intradermal, intramammary, intravenous injections and other administrative methods known in the art. Implants include intra artherial implants, for example, an intrahepatic artery implant.

Any of the combinations of a morpholinyl anthracycline derivative having formula (I) as defined above, and a demethylating agent are intended as fixed combination and for simultaneous, separate, or sequential use.

By the term “antineoplastic effect”, as used herein, it is meant the inhibition of the growth tumor, preferably the complete regression of the tumor, by administering an effective amount of the combination comprising a morpholinyl anthracycline derivative having formula (I), and a demethylating agent.

A further aspect of the present invention relates to the a combination of a morpholinyl anthracycline derivative having formula (I), as defined above, and a demethylating agent for the prevention or treatment of metastasis or the treatment of tumors by inhibition of angiogenesis.

The constituents of the combined preparations according to the invention can be administered to a patient in any acceptable manner that is medically acceptable including orally, parenterally, or with local therapeutic approaches such as, e.g., implants. Oral administration includes administering the constituents of the combined preparation in a suitable oral form such as, e.g., tablets, capsules, lozenges, suspensions, solutions, emulsions, powders, syrups and the like. Parenteral administration includes administering the constituents of the combined preparation by subcutaneous, intravenous or intramuscular injections. Local therapeutic approaches include implants, for example intra-arterial implants.

Typically a morpholinyl anthracycline derivative having formula (I) is administered intravenously, typically a demethylating agent is administered intravenously or orally. The actual preferred dosage, method, order and time of administration of the constituents of the combined preparations of the invention may vary according to, inter alia, the particular pharmaceutical formulation of a morpholinyl anthracycline derivative having formula (I), being utilized and the particular pharmaceutical formulation of a demethylating agent being utilized, the particular cancer being treated, the age, condition, sex and extent of the disease treated and can be determined by one of skill in the art.

The dosage regimen must therefore be tailored to the particular of the patient's conditions, response and associate treatments, in a manner, which is conventional for any therapy, and may need to be adjusted in response to changes in conditions and/or in light of other clinical conditions.

As a non limiting example, suitable dosages of the morpholinyl anthracycline derivative of formula (I) may range from about 0.05 mg/m² to about 100 mg/m² of body surface area and, more preferably, from about 0.1 to about 10 mg/m² of body surface area.

For the administration of the demethylating agent, according to the method of the invention, the course of therapy generally employed is that generally used for this kind of drugs.

For example, 5′aza-2′-deoxycytidine may be administered at doses varying from about 5 mg/day to about 2.000 mg/day and, more preferably, from about 50 to about 400 mg/day.

As a further examples, valproic acid may be administered at doses varying from about 5 mg/day to about 1.000 mg/day and, more preferably, from about 10 to about 250 mg/day; procanamide may be administered at doses varying from about 100 mg/day to about 10.000 mg/day and, more preferably, from about 500 to about 4.000 mg/day. When the active constituents of the combined preparation according to the invention are supplied along with a pharmaceutically acceptable carrier or excipient, a pharmaceutical composition is formed. Such pharmaceutical composition constitutes a further embodiment of the invention.

Pharmaceutically acceptable carriers and excipients are chosen such that side effects from the pharmaceutical compound are minimized and the performance of the compound is not cancelled or inhibited to such an extent that treatment is ineffective. Pharmaceutically acceptable carriers or excipients to be utilized in the preparation of a pharmaceutical composition according to the invention are well known to people skilled in the art of formulating compounds in a form of pharmaceutical compositions. For example, “pharmaceutically acceptable carrier” refers to one or more compatible solid or liquid filler, diluent or encapsulating substances which are suitable for administration to mammals including humans. For example, “pharmaceutically acceptable excipient” refers to any inert substance used as a diluent or vehicle for an active substance(s) that is intentionally added to the formulation of a dosage form.

The term includes binders, fillers' disintegrants, and lubricants.

Nevertheless, the combination of the present invention can be employed without adding any sustained-release adjuvant.

Techniques for formulation and administration of drugs can be found in “Remington's Pharmacological Sciences”; Mack Publishing Co., Easton, Pa., latest edition.

Pharmaceutical compositions suitable for parenteral administration are formulated in a sterile form. The sterile composition thus may be a sterile solution or suspension in a non-toxic parenterally acceptable diluent or solvent.

The amount of an active ingredient contained in the pharmaceutical composition according to the invention may vary quite widely depending upon many factors such as, for example, the administration route and the vehicle.

As an example, the pharmaceutical compositions of the invention may contain from about 0.05 mcg to about 100 mcg of a substituted morpholinyl anthracycline derivative of formula (I) as defined above; and from 1 mg to 10,000 mg of a demethylating agent.

Pharmaceutical compositions according to the invention are useful in anticancer therapy.

The present invention further provides a commercial kit comprising, in a suitable container means, a morpholinyl anthracycline derivative of formula (I), as defined above, and a demethylating agent. In a kit according to the invention a morpholinyl anthracycline derivative of formula (I), as defined above, and a demethylating agent are present within a single container means or within distinct container means.

Another embodiment of the present invention is a commercial kit comprising a pharmaceutical composition as described above.

Kits according to the invention are intended for simultaneous, separate or sequential use in antitumor therapy.

Kits according to the invention are intended for use in anticancer therapy.

The antineoplastic effect of the combined preparations of the present invention is shown, for instance, by the following in vitro tests, which are intended to illustrate the present invention without posing any limitation to it.

In Vitro Antitumor Efficacy of Nemorubicin in Cells Treated with 5-Aza-2′-Deoxycytidine

The effect of 5-aza-2′-deoxycytidine on the cytotoxic activity of morpholinyl anthracycline derivative (nemorubicin) was tested on a murine leukaemia cell line (L1210 cells). Cells were grown in RPMI 1640 medium supplemented with 10% foetal calf serum. Exponentially growing cells were seeded and exposed to 5-aza-2′-deoxycytidine immediately after seeding for 6 days, so as to allow efficient demethylation of the CpG islands. 5-Aza-2′-deoxycytidine inhibited DNA methylation by reducing the biochemical activity of DNA methyltransferase via the formation of a covalent complex with this enzyme; this is believed to deplete methyltransferase activity, hence resulting in DNA demethylation (Goffin J. and Eisenhauer E. Annals of Oncology 13:1699-1716, 2002). Six days following 5-aza-2′-deoxycytidine treatment, cells were diluted and treated with different concentrations of nemorubicin (50, 100, 200 and 500 nM) in comparison with cells not pretreated with the demethylating agent 5-aza-2′-deoxycytidine. In vitro drug sensitivity was determined by counting surviving cells by a Coulter Counter apparatus. The antiproliferative activity of the drug was evaluated after 24 h treatment and calculated from dose-response curves and expressed as dose inhibiting 50% cell growth (IC₅₀). IC₅₀ values were calculated from two-three independent experiments each consisting of at least three replicates. Combination indices (CI) were calculated using a computer program for multiple drug effect analysis based on the equation of Chou-Talalay (Adv Enzyme Regul 1984; 22:27-55) for mutually non-exclusive drugs. CI values <0.3-0.8 are indicative of synergism, 0.8-1.2 are indicative of additivity, 1,3>3 are indicative of antagonism.

The growth inhibitory activity of nemorubicin observed in these conditions expressed as IC₅₀ and CI are reported in Table 1.

TABLE 1
Cytotoxic effect of nemorubicin on L1210 cells with
or without pre-treatment with 5-aza-2′-deoxycytidine.

| Cell line | IC₅₀ (nM) | CI |
|---|---|---|
| L1210 cells pretreated with 5- | 130 | 0.14 |
| aza-2′-deoxycytidine | | |
| L1210 cells untreated | 210 | |

As a result, the pre-treatment with 5-aza-2′-deoxycytidine increased the cytotoxicity of nemorubicin (IC₅₀ with and without 5-aza-2′-deoxycytidine: 130 and 210 nM respectively). Based on resultant CI value, the combination nemorubicin and the demethylating agent 5-aza-2′-deoxycytidine has a synergistic effect.

We further evaluate the effect of this combination on tumor cells resistant to nemorubicin. As shown here below, the treatment of L1210 cells resistant to nemorubicin (L1210/MMRDX cells) with 5-aza-2′-deoxycytidine is able to reverse the resistance to nemorubicin making resistant cells as sensitive to the effect of nemorubicin as the parental ones.

In Vitro Antitumor Efficacy of Nemorubicin in Cells Resistant to Nemorubicin, Treated with 5-Aza-2′-Deoxycytidine

The effect of 5-aza-2′-deoxycytidine on the cytotoxic activity of nemorubicin was tested in comparison on L1210 cells and on the strain resistant to nemorubicin (L1210/MMRDX) (Geroni C. et al, Br J Cancer 9:315-19, 1994). Cells were grown in RPMI 1640 medium supplemented with 10% foetal calf serum. Exponentially growing cells were seeded and exposed to 5-aza-2′-deoxycytidine and nemorubicin as reported here above. In vitro drug sensitivity was determined by counting surviving cells by a Coulter Counter apparatus. The antiproliferative activity of the drug was evaluated after 24 h treatment and calculated from dose-response curves and expressed as IC₅₀. IC₅₀ values were calculated from two-three independent experiments each consisting of at least three replicates. Combination indices (CI) were calculated as described above. The growth inhibitory activity of nemorubicin observed in these conditions expressed as IC₅₀ and CI are reported in Table 2.

TABLE 2
Cytotoxic effect of nemorubicin on L1210 and L1210/MMRDX
cells pretreated with 5-aza-2′-deoxycytidine.

| Cell line | IC₅₀ (nM) | CI |
|---|---|---|
| L1210/MMRDX cells pretreated with | 140 | 0.06 |
| 5-aza-2′-deoxycytidine | | |
| L1210/MMRDX cells untreated | 480 | |
| L1210 cells pretreated with 5-aza-2′- | 130 | 0.14 |
| deoxycytidine | | |
| L1210 cells untreated | 210 | |

As a result, the pre-treatment with 5-aza-2′-deoxycytidine increases the antitumor activity of nemorubicin on L1210/MMRDX cells (IC₅₀ with and without 5-aza-2′-deoxycytidine: 140 and 480 nM respectively). Based on resultant CI value, there is a strong synergistic effect of this combination.

In addition, when the cells were pretreated with 5-aza-2′-deoxycytidine, the effective dose in the resistant strain is comparable to that in the sensitive ones (IC₅₀ L1210/MMRDX and L1210: 140 and 130 nM, respectively), hence indicating e reversion of nemorubicin resistance through the combination with a demethylating agent.

1-16. (canceled)

17. A combination comprising a morpholinyl anthracycline derivative having formula (I): 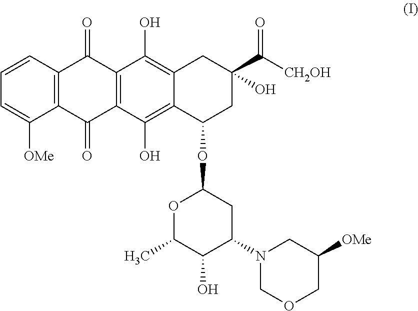
or a pharmaceutically acceptable salt thereof and a demethylating agent, having an antineoplastic effect.

18. A combination according to claim 17, wherein the morpholinyl anthracycline of formula (I) is nemorubicin hydrochloride.

19. A combination according to claim 17, wherein the demethylating agent is 5′aza-cytidine, 5-aza-2′-deoxycytidine (decitabine), zebularine [1-(β-D-ribofuranosyl(dihydro-pyrimidin-2-1)], L-methionine, inhibitors of histone deacetylase (HDAC) such as, for instance, valproic acid or trichostatin A, apicidine, hydralazine, procainamide (pronestyl), antisense oligonucleotides directed against DNA methyltransferase messeger RNA, their admixtures and derivatives thereof.

20. A combination according to claim 17 for the treatment of hyperproliferative diseases such as cancer.

21. A combination according to claim 17 for reversing the resistance to nemorubicin in cells resistant thereto.

22. A pharmaceutical composition comprising a combination as defined in claim 17 admixed with a pharmaceutically acceptable carrier, diluent or excipient.

23. A pharmaceutical composition as defined in claim 22 for the treatment of hyperproliferative diseases such as cancer.

24. A pharmaceutical composition as defined in claim 22 for reversing the resistance to nemorubicin in cells resistant thereto.

25. A pharmaceutical product comprising a morpholinyl anthracycline derivative as defined in claim 17 and a demethylating agent as combined preparation for simultaneous, sequential or separate use in therapy.

26. A method for the treatment of hyperproliferative diseases such as cancer or for reversing the resistance to nemorubicin in cells resistant thereto, which method comprises the administration to a warm-blooded animal such as man that is in need of such treatment of effective amounts of the combination as defined in claim 17.

27. The method of claim 26 wherein the administration of the combination is simultaneous, sequential or separate.

28. A commercial kit comprising, in a suitable container mean, a morpholinyl anthracycline derivative as defined in claim 17, and a demethylating agent.

29. A commercial kit comprising a pharmaceutical composition or product as defined in claim 23.

30. A commercial kit according to claim 28 for simultaneous, separate or sequential use in antitumor therapy.

31. A commercial kit according to claim 29 for simultaneous, separate or sequential use in antitumor therapy.
